(19) United States
(12) Patent Application Publication
(10) Pub. No.: US 2006/0030771 A1
(43) Pub. Date: Feb. 9, 2006

(54) System and method for sensor integration

(76) Inventors: Lewis Levine, Weston, MA (US); Peter Traneus Anderson, Andover, MA (US); Thomas Peterson, Wilmington, MA (US)

Correspondence Address: MCANDREWS HELD & MALLOY, LTD, 500 WEST MADISON STREET, SUITE 3400, CHICAGO, IL, 60661, US

(21) Appl. No.: 10/911,153

(22) Filed: Aug. 3, 2004

(51) Int. Cl. A61B 5/05

(52) U.S. Cl. 600/424

(57) ABSTRACT

A system and method for sensor integration includes a medical device, a transmitter attached to the medical device, and a receiver. The medical device can include a tracking point. The transmitter can be connected to the medical device to minimize a distance between the tracking point and the transmitter. The transmitter can transmit a position signal. The receiver can receive the position signal. The signal may be employed to determine at least one of a position and orientation of the transmitter relative to the receiver. The transmitter may include electronic circuitry capable of measuring additional telemetry information and transmitting the telemetry in a telemetry signal. The transmitter may also transmit an identity signal.

### BACKGROUND OF THE INVENTION

The present invention generally relates to an electromagnetic tracking system. In particular, the present invention relates to a system and method for sensor integration in an electromagnetic tracking system.

Medical practitioners, such as doctors, surgeons, and other medical professionals, often rely upon technology when performing a medical procedure, such as image-guided surgery (“IGS”) or examination. An IGS system may provide positioning and/or orientation (“P&O”) information for the medical instrument with respect to the patient or a reference coordinate system, for example. A medical practitioner may refer to the IGS system to ascertain the P&O of the medical instrument when the instrument is not within the practitioner's line of sight with regard to the patient's anatomy, or with respect to non-visual information relative to the patient. An IGS system may also aid in pre-surgical planning.

The IGS or navigation system allows the medical practitioner to visualize the patient's anatomy and track the P&O of the instrument. The medical practitioner may use the tracking system to determine when the instrument is positioned in a desired location or oriented in a particular direction. The medical practitioner may locate and operate on, or provide therapy to, a desired or injured area while avoiding other structures. Increased precision in locating medical instruments within a patient may provide for a less invasive medical procedure by facilitating improved control over smaller, flexible instruments having less impact on the patient. Improved control and precision with smaller, more refined instruments may also reduce risks associated with more invasive procedures such as open surgery.

The highly accurate tracking technology found in navigation systems may also be used to track the P&O of items other than medical instruments in a variety of applications. That is, a tracking system may be used in other settings where the P&O of an object in an environment is difficult to accurately determine by direct or indirect inspection. For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. A receiver detects the response signal. The signal emitted by the transponder may then be used to monitor the position of a person or object in a simulated environment.

Tracking systems may be optical, ultrasonic, inertial, or electromagnetic, for example. Electromagnetic tracking systems may employ coils as receivers and transmitters. Typically, an electromagnetic tracking system is configured in an industry-standard coil architecture (“ISCA”). The ISCA is characterized by three colocated orthogonal quasi-dipole transmitter coils and three colocated quasi-dipole receiver coils. Such a configuration currently appears in many products such as the Polhemus FASTRACK®, for example. Other systems may use three large, non-dipole, non-colocated transmitter coils with three colocated quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space.

The ISCA tracker architecture uses a three-axis quasi-dipole coil transmitter and a three-axis quasi-dipole coil receiver. Each three-axis transmitter or receiver is built so that the three coils exhibit the same effective area, are oriented orthogonal to one another, and are centered at the same point. If the coils are small enough compared to a distance between the transmitter and receiver, then the coil may exhibit dipole behavior. Magnetic fields generated by the trio of transmitter coils may be detected by the trio of receiver coils. Using three approximately concentrically positioned transmitter coils and three approximately concentrically positioned receiver coils, for example, nine parameter measurements may be obtained. From the nine parameter measurements and one known position or orientation parameter, a position and orientation calculation may determine position and orientation information for each of the transmitter coils with respect to the receiver coil trio with three degrees of freedom.

In medical and surgical imaging, such as intraoperative or perioperative imaging, images are formed of a region of a patient's body. The images are used to aid in an ongoing procedure with a surgical tool or instrument applied to the patient and tracked in relation to a reference coordinate system formed from the images. Image-guided surgery is of a special utility in surgical procedures such as brain surgery and arthroscopic procedures on the knee, wrist, shoulder or spine, as well as certain types of angiography, cardiac procedures, interventional radiology and biopsies in which x-ray images may be taken to display, correct the P&O of, or otherwise navigate a tool or instrument involved in the procedure.

Several areas of surgery involve very precise planning and control for placement of an elongated probe or other device in tissue or bone that is internal or difficult to view directly. In particular, for brain surgery, stereotactic frames that define an entry point, probe angle and probe depth are used to access a site in the brain, generally in conjunction with previously compiled three-dimensional diagnostic images, such as MRI, PET or CT scan images, which provide accurate tissue images. For placement of pedicle screws in the spine, where visual and fluoroscopic imaging cannot capture an axial view to center a profile of an insertion path in bone, navigation systems have also been useful.

When used with existing CT, PET or MRI image sets, previously recorded diagnostic image sets define a three dimensional rectilinear coordinate system, either by virtue of their precision scan formation or by the spatial mathematics of their reconstruction algorithms. However, it may be desirable to correlate the available fluoroscopic views and anatomical features visible from the surface or in fluoroscopic images with features in the 3-D diagnostic images and with external coordinates of instruments or devices being employed. Correlation is often done by providing implanted fiducials and adding externally visible or trackable markers that may be imaged. Using a keyboard or mouse, or algorithmically via sophisticated image processing techniques, fiducials may be identified in the various images. Thus, common sets of coordinate registration points may be identified in the different images. The common sets of coordinate registration points may also be trackable in an automated way by an external coordinate measurement device, such as a suitably programmed off-the-shelf optical tracking assembly. Instead of imageable fiducials, which may for example be imaged in both fluoroscopic and MRI or CT images, such systems may also operate to a large extent with simple optical tracking of the surgical tool and may employ an initialization protocol wherein a surgeon touches or points at a number of bony prominences or other recognizable anatomic features in order to define external coordinates in relation to a patient anatomy and to initiate software tracking of the anatomic features.

Other forms of data exhibiting three-dimensional spatial characteristics include, but are not limited to, maps of cortical excitation/response data, cardiac wall motion studies, or temporal maps of anatomical changes with respect to disease or developmental processes. When correlated to the frame of reference of the patient, an IGS system can be used to navigate these other forms of spatial data with respect to image data, providing an augmented “view” of the patient's condition.

Generally, image-guided surgery systems operate with an image display which is positioned in a surgeon's field of view and which displays a few panels such as a selected MRI image and several x-ray or fluoroscopic views taken from different angles. Three-dimensional diagnostic images typically have a spatial resolution that is both rectilinear and accurate to within a very small tolerance, such as to within one millimeter or less. By contrast, fluoroscopic views may be distorted. The fluoroscopic views are shadowgraphic in that they represent the density of all tissue through which the conical x-ray beam has passed. In tool navigation systems, the display visible to the surgeon may show an image of a surgical tool, biopsy instrument, pedicle screw, probe or other device projected onto a fluoroscopic image, so that the surgeon may visualize the orientation of the surgical instrument in relation to the imaged patient anatomy. An appropriate reconstructed CT or MRI image, which may correspond to the tracked coordinates of the probe tip, may also be displayed.

Among the systems that have been proposed for effecting such displays, many rely on closely tracking the position and orientation of the surgical instrument in external coordinates. The various sets of coordinates may be defined by robotic mechanical links and encoders, or more usually, are defined by a fixed patient support, two or more receivers such as video cameras which may be fixed to the support, and a plurality of signaling elements attached to a guide or frame on the surgical instrument that enable the position and orientation of the tool with respect to the patient support and camera frame to be automatically determined by triangulation, so that various transformations between respective coordinates may be computed. Three-dimensional tracking systems employing at least two video cameras and a plurality of emitters or other position signaling elements have long been commercially available and are readily adapted to such operating room systems. Similar systems may also determine external position coordinates using commercially available acoustic ranging systems in which three or more acoustic emitters are actuated and their sounds detected at plural receivers to determine their relative distances from the detecting assemblies, and thus define by simple triangulation the position and orientation of the frames or supports on which the emitters are mounted. When tracked fiducials appear in the diagnostic images, it is possible to define a transformation between patient coordinates and the coordinates of the image.

Current tracking systems require a large number of components, especially sensors. However, an increase in the number of components in a tracking system interferes with medical procedures, especially those procedures requiring reduced “clutter” in the operating or tracking environment.

Furthermore, current systems and methods employ placing a tracking sensor on a medical instrument of a known size and shape. The instrument is calibrated by determining the distance between the sensor and the various extremities of the instrument. During a medical procedure, the location of the instrument extremities is calculated by determining the known location of the sensor and combining this location with the measured distance between the sensor and the instrument extremities. However, due to instrument distortion such as flex, for example, the measured distance between the sensor and instrument extremities can change during the medical procedure. This distortion can then cause decreased accuracy in determining the location of instrument extremities.

In addition, current sensors are limited in functionality. For example, current sensors are generally single-use sensors unable to provide, in addition to telemetry data, other valuable information such as identification information, vital statistics and other physical data such as pressure, temperature, force, deflection, stress or strain.

Thus, a need exists for a navigation system and method employing a tracking technology that may be integrated into devices so as to increase the accuracy, reliability, and ease-of-use of the system. Moreover, the integration of sensors in existing or new medical instruments at more optimum locations increases the accuracy of determining P&O. In addition, providing sensors that include increased functionality (such as, for example, providing identification information, vital statistics and other physical data) allow a tracking system to collect additional valuable information. Such a system and method providing for fewer components in a tracking or operating environment also can reduce the overall cost of the system, but also decrease the amount of “clutter” interfering with the safe and effective operating or tracking environment.

### BRIEF SUMMARY OF THE INVENTION

The present invention provides a sensor integration system. The system includes a medical device, a transmitter and a receiver. The medical device includes a tracking point. The transmitter is connected to the device so as to minimize a distance between the tracking point and the transmitter. The transmitter transmits a position signal. The receiver receives the position signal.

The present invention also provides a method for integrating a sensor into a medical device. The method includes connecting a transmitter to a medical device, transmitting a position signal, and receiving the position signal. The transmitter is attached to the medical device to minimize a distance between a tracking point of the device and the transmitter. The transmitter transmits the position signal. The receiver receives the position signal.

The present invention also discloses a method for locating a medical device in a patient and providing a device characteristic. The method includes connecting a transmitter to the medical device, transmitting a signal from the transmitter, and receiving the signal at a receiver. The signal includes at least one of a position and orientation of the transmitter relative to the receiver and the device characteristic.

### BRIEF DESCRIPTION OF SEVERAL VIEWS OF THE DRAWINGS

FIG. 1 illustrates a tracking system used in accordance with an embodiment of the present invention.

FIG. 2 depicts a flowchart for a method for the integration of sensors in medical devices, used in accordance with an embodiment of the present invention.

### DETAILED DESCRIPTION OF THE INVENTION

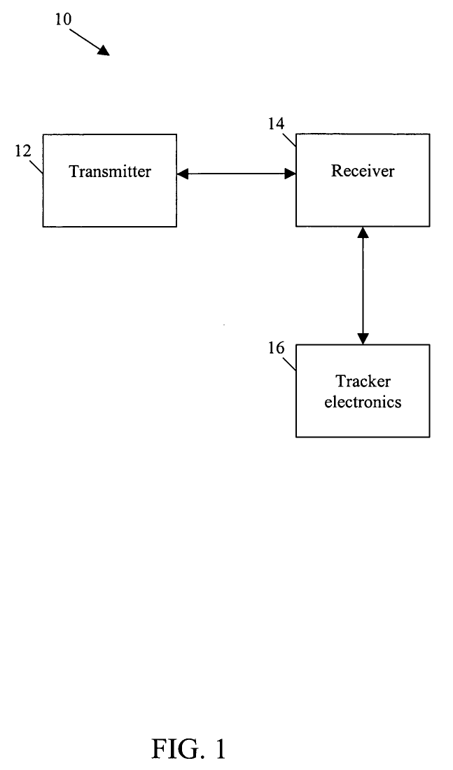

FIG. 1 illustrates a tracking system 10 used in accordance with an embodiment of the present invention. The tracking system 10 includes at least one transmitter 12, a receiver 14 and tracker electronics 16. Transmitter 12 transmits a signal. Receiver 14 detects the signal. Tracker electronics 16 analyze the received signal. Using transmitter 12 and receiver 14, at least one of a position and orientation of transmitter 12 may be tracked. Tracking system 10 may be configured in ISCA, for example.

In an embodiment, transmitter 12 may be a wireless transmitter. For example, transmitter 12 may be a wireless ISCA transmitter. In another embodiment, transmitter 12 may be a wired transmitter. Transmitter 12 may also be a sensor including additional electronics and capable of transmitting a signal through another object, such as a medical instrument or a combination of a medical instrument and a human body. For example, transmitter 12 may be a sensor employing a gyroscope or accelerometer.

Transmitter 12 can be connected to a medical device, such as a medical instrument or implant. For example, transmitter 12 may be attached to a tip of a reducing rod, a drill bit, debrider blade, or a guidewire. Similarly, transmitter 12 may be attached to an artificial hip or knee implant. Transmitter 12 may be added to an existing device by wrapping a wire coil around a component of the device where tracking is desired to create an EM coil, for example. In order to reduce interference from the material of the device, transmitter 12 may be partially formed by wrapping a wire coil around the device, thereby protecting the EM field from a source of interference.

Transmitter 12 may be connected to the instrument or implant by embedding transmitting in the instrument or implant. For example, during the production of a reducing rod, drill bit, guidewire, artificial hip or artificial knee, pedicle screw, artificial disk, or the like, transmitter 12 may be embedded into materials used to create the instrument or implant. By embedding transmitter 12, it may be fixed in a given location within a device and therefore be resistant to movement independent of the device. However, other factors may require the embedding of transmitter 12 into a medical device.

A wireless transmitter 12 may draw power from an instrument on which transmitter 12 is attached or embedded or may have a separate power source, for example. However, use of a battery as a power source may result in interference to system 10. In order to reduce this interference, transmitter 12 may be partially formed by wrapping a wire coil around the power source, or battery, thereby protecting the EM field from the source of interference. Other sources of energy for transmitter 12 may include induction or piezoelectric generation, for example.

In an embodiment, receiver 14 includes receiver dipole coils or coil trios. Receiver 14 may also include a greater or fewer numbers of coils. Receiver 14 may include an array of coils capable of receiving telemetry and/or P&O data transmitted by transmitter 12. For example, receiver 14 may be a twelve-coil wired EM receiver.

In an embodiment, tracker electronics 16 include a computer processor capable of determining a P&O of transmitter 12 relative to a reference point based on a signal received from receiver 14. For example, tracker electronics 16 may include the GE InstaTrak®. Other examples of tracker electronics may include any one of the Liberty™, Patriot™, or FASTRAK™ products produced by Polhemus. The connection between receiver 14 and tracker electronics 16 may be a wired or wireless connection. Tracker electronics 16 may also be integrated with receiver 14 or may be a separate module, for example.

In an embodiment, the signal transmitted by transmitter 12 can include tracking data, for example. Tracking data can include the P&O (position and/or orientation) of transmitter 12 relative to receiver 14. Tracking data may be determined by receiver 14 receiving a current through an attached wire or power source and generating a magnetic field, for example. Mutual inductance may then be used to identify positions and/or orientations of transmitter 12 in the system 10. Electromagnetic coils in transmitter 12 detect the magnetic field and transmitter 12 may communicate a signal proportional to the strength of the magnetic field to receiver 14, for example. Receiver 14 may communicate the received signal to tracker electronics 16. Tracker electronics 16 may then measure the received signal and calculate the P&O of transmitter 12 relative to receiver 14, for example.

As transmitter 12 may be placed or embedded at various locations on an existing medical instrument or implant, calibration of system 10 may become much simpler and more accurate. Current tracking systems require calibration of three points in space, namely a transmitter location, a receiver location and a device (such as an instrument or implant) location. For example, in the calibration of a tracking system for an elongated medical instrument, such as a reducing rod, screw, debrider blade, or guidewire, typically the transmitter is located on the instrument at a point a known or measured distance away from the end of the instrument inserted into a patient. For example, a reducing rod may have an insertion tip that is inserted into the patient and an opposite end where an operator holds and maneuvers the rod. Conventional tracking systems place the transmitter near the opposite end of the reducing rod. The distance between the location of the transmitter and the insertion tip is either known or subsequently measured. The conventional tracking system then determines the P&O of the transmitter relative to a receiver. The location of the insertion tip is then estimated by adding the distance between the transmitter and insertion tip to the position of the transmitter.

Conversely, in the present system, as transmitter 12 may be placed at, or manufactured into, virtually any location on an instrument or implant, for example, calibration of system 10 may not require the estimation of any distance between transmitter 12 and a point of interest, such as a tip or end of a medical device. Transmitter 12 can be located so as to minimize the distance between transmitter 12 and the point of interest. For example, the point of interest can be a tracking point, such as a tip of a reducing rod to be inserted into a patient or a point on a hip implant. In this way, the point of interest may become a point of the instrument or implant that is tracked, for example.

Transmitter 12 can be attached or embedded in the medical device at the point of interest. However, due to physical, structural and electrical limitations, among others, transmitter 12 may not always be capable of being attached or embedded at the point of interest. Therefore, by minimizing the distance between transmitter 12 and a point of interest, two points may be used to calibrate system 10 (namely positions of transmitter 12 and receiver 14), whereas three points are required to calibrate conventional tracking systems (namely positions of a transmitter, a receiver and a distance between the transmitter and point of interest), as described above.

For example, transmitter 12 may be attached or embedded in the tip of a surgical drill bit. As the drill bit is used to bore into a patient's bone, system 10 can track the location of the drill bit tip by tracking the P&O of transmitter 12 relative to receiver 14. In this way, a surgeon may know, at all times, how far into the patient the drill bit has progressed. As the surgeon is able to track the P&O of the transmitter 12 (and therefore the drill bit tip), flex of the drill bit may not affect (or have a decreased effect on) the tracking of the location of the drill bit tip.

In another example, system 10 may be useful in navigating surgical guidewire in a patient. Similar to above, transmitter 12 may be attached or embedded in the insertion tip of the guidewire, for example. The tip may then be inserted and moved through a patient while system 10 is able to track the P&O of transmitter 12 and therefore the insertion tip. Any flex of the guidewire may not affect (or have a decreased effect on) the tracking of the tip's P&O as system 10 is tracking the P&O of transmitter 12 and therefore the insertion tip (or a point near the tip), and not a distant point relative to the tip. Once the guidewire has been properly placed in the patient, an implant may be inserted into the patient over the guidewire. In this way, system 10 may provide for increased accuracy in the insertion of implants.

Transmitter 12 may broadcast P&O information (or any other information, as described below) continuously. For example, transmitter 12 may broadcast a signal to receiver 14 in a continuous manner. Receiver 14 may then continuously receive the signal and tracker electronics 16 may continuously measure or determine the P&O of transmitter 12, for example.

In another embodiment, transmitter 12 may broadcast P&O information (or any other information, as described below) on an at-demand basis. On an at-demand basis, transmitter 12 can broadcast a signal when receiving an other signal from an outside entity, such as receiver 14, for example. The receiver 14 may therefore send a “ping” signal to transmitter 12, for example. Once transmitter 12 receives the “ping” signal, transmitter 12 may respond with a signal containing P&O information, for example. Receiver 14 may then receive P&O information as described above, for example, when receiver 14 makes a demand for such information.

In another embodiment, transmitter 12 may broadcast P&O information (or any other information, as described below) on a regular or cyclic basis. In a regular or cyclic basis, transmitter 12 may transmit a signal at regular time intervals. For example, transmitter 12 may transmit a signal once every three seconds. Receiver 14 may therefore receive the signal on a periodic, three second interval basis, for example.

Transmitter 12 may also provide telemetry other than or in addition to a P&O of transmitter 12 relative to receiver 14. For example, transmitter 12 may include additional electronic circuitry capable of determining additional data to be transmitted, such as a pH reading, pressure, stress and/or strain to the device, temperature or any other vital statistics, such as a pulse. In order to determine the additional data, the additional electronic circuitry may include a printed circuit board (“PCB”), for example.

Transmitter 12 may also transmit information other than P&O and/or telemetry. For example, transmitter 12 may transmit a signal including a unique identifier to receiver 14. The identification signal may include information or data related to the instrument or implant to which transmitter 12 may be attached. For example, transmitter 12 may broadcast a signal that identifies a type of guidewire to which transmitter 12 is attached. The identification information may include any information useful to discern a type of instrument or implant or an identity of a manufacturer, patient or host, for example. The identification signal may be created by circuitry external to transmitter 12, as described above, or the identification signal may be unique to the data. For example, an identification signal used to identify an implant created by a first manufacturer may differ in any one of frequency or amplitude from an implant created by a second manufacturer.

In general, any one of the above telemetry and identification signals may be considered as device characteristic signals. For example, a transmitter 12 that determines a pH reading and transmits the reading (in addition to P&O data) to receiver 14 is transmitting both P&O data and a device characteristic signal. In another example, the device characteristic signal may include information unique to the transmitter 12 or the device to which transmitter 12 is attached, as described above. For example, the device characteristic signal may include any information useful to discern a type of instrument or implant or an identity of a manufacturer, patient or host, as described above.

Transmitter 12 may broadcast the additional telemetry or identification signals using a modulated signal. Using a modulated signal may allow transmitter 12 to transmit P&O information to receiver 14 while other telemetry or identity data may be modulated with the P&O signal.

In another embodiment, transmitter 12 may broadcast additional telemetry and/or identity signals on a cyclic basis. For example, transmitter 12 may cycle through the transmission of P&O data, followed by first telemetry data (for example, a pH reading), followed by identity data (for example, an identity of a manufacturer), followed by P&O data, first telemetry data, identity data, and so on.

Transmitter 12 may use any number of means to multiplex the signal data (as described above) as is commonly known in the art, such as in the time or frequency domains. The received signal may then be de-multiplexed by tracker electronics 16 so as to separate the data components for further processing. For example, transmitter 12 may transmit any one or more of P&O data, telemetry, and identification data in a multiplexed fashion. After being received, the multiplexed signal may then be de-multiplexed by tracker electronics 16 into the various components of the signal.

In another embodiment, transmitter 12 may broadcast any information, including P&O information, identity information and/or additional telemetry on a duty cycle basis. A duty cycle basis may include transmitter 12 cycling between the transmissions of a signal to receiver 14 and transmitting a duty signal to act on an object. A duty signal may include a signal transmitted by transmitter 12 that acts on a patient. For example, a duty signal may be transmitted to apply an electrical pulse or signal, or a radiofrequency signal to act on tissue in a patient.

For example, transmitter 12 may be used in conjunction with a catheter to perform radiofrequency ablation of a heart. Conventional radiofrequency ablation includes a physician guiding a catheter with an electrode inside a chamber of a heart. Typically, the physician guides the catheter using fluoroscopic images of the patient's chest area. The physician then transmits radiofrequency energy through the catheter and electrode to destroy heart muscles causing an irregular heartbeat in a given area. As conventional tracking systems suffer from inaccurate determination of the exact P&O of an instrument tip, as described above, the physician's placement of the electrode may be hampered by improper placement.

System 10 may be used to perform radiofrequency ablation using a transmitter 12 duty cycle, for example. Transmitter 12 may be placed on a catheter tip and serve both as a tracking sensor and an electrode, for example. As a physician moves the catheter into a patient's heart, system 10 may determine the P&O of transmitter 12 and therefore the catheter tip and electrode. Once transmitter 12 is inside the patient's heart, transmitter 12 may cycle between transmitting P&O data and/or telemetry data to receiver 14 and transmitting radiofrequency energy to destroy heart muscles, for example.

In addition to the above example, transmitter 12 may also measure electrical signals inside the patient's heart. For example, transmitter 12 may cycle between transmitting P&O information and transmitting a measured electrical signal in the heart. In this way, a physician may be able to more accurately map out electrical signals inside a patient's heart, thereby allowing for increased accuracy when ablating the heart. Moreover, transmitter 12 may cycle between transmitting P&O information, measuring an electrical signal of the heart, and applying radiofrequency energy to areas of the heart where the measured electrical signal exceeds a given threshold, for example. Transmitter 12 may measure signals on a continuous, on-demand or cyclic basis, as described above.

System 10 can be applicable in environments other than the tracking of medical devices, instruments and implants. For example, system 10 may be employed in any environment where a sensor providing information when requested or on a cyclic basis would be desired. In an embodiment, system 10 may be employed in a security setting (for example, in airport security screenings). Security personnel employing system 10 may therefore track transmitters 12 located inside a person. System 10 may then be able to determine whether a security alarm occurs because the person is concealing a weapon or whether the person has a medical device or implant inside his or her body, for example. As described above, transmitter 12 may be configured to provide identity information that provides receiver 14 with information regarding the type of implant or device, for example.

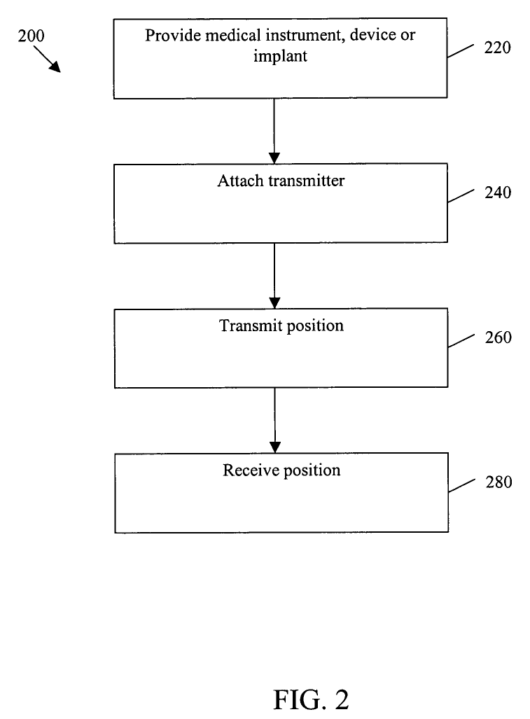

FIG. 2 depicts a flowchart for a method 200 for the integration of sensors in medical devices, used in accordance with an embodiment of the present invention.

First, at step 220, a medical device is provided (for example, a medical instrument or implant), as described above. For example, a catheter may be provided for a heart ablation procedure.

Next, at step 240, a transmitter is attached to the device, as described above. The device is then employed in a medical procedure. For example, a hip implant may be implanted into a patient, a reducing rod may be inserted into a bone, or a catheter may be inserted into a patient's heart.

In another embodiment, at step 240 the transmitter may be embedded in the device, as described above. For example, the transmitter may be embedded in the implant during the manufacture of the implant.

Next, at step 260 the transmitter transmits or broadcasts P&O information, as described above. In another embodiment, also as described above, the transmitter may transmit or broadcast other information, such as identity information or other telemetry information. In another embodiment, the transmitter may transmit multiple signals on a cyclic basis, as described above. In another embodiment, the transmitter may also transmit a signal or energy to the patient, such as in a duty cycle as described above.

Next, at step 280, a receiver receives the signal transmitted by the transmitter, as described above.

In another embodiment, after step 280, the method may proceed to step 260 to transmit P&O information, as described above. In this way, the method may proceed in a cyclic manner by continuously transmitting and receiving P&O information.

While particular elements, embodiments and applications of the present invention have been shown and described, it is understood that the invention is not limited thereto since modifications may be made by those skilled in the art, particularly in light of the foregoing teaching. It is therefore contemplated by the appended claims to cover such modifications and incorporate those features that come within the spirit and scope of the invention.

What is claimed is:

1. A sensor integration system including:
a medical device including a tracking point;
a transmitter connected to said medical device to minimize a distance between said point and said transmitter, said transmitter transmitting a position signal; and
a receiver receiving said position signal.

2. The system of claim 1, wherein said transmitter is embedded in said medical device.

3. The system of claim 1, further including tracking electronics measuring said position signal and determining at least one of a position and orientation of said transmitter relative to said receiver.

4. The system of claim 1, wherein said position signal includes at least one of a unique identifier and telemetry signal.

5. The system of claim 1, wherein said transmitter is an electromagnetic coil and said receiver is an array of electromagnetic coils.

6. The system of claim 1, wherein said transmitter includes electronic circuitry capable of measuring additional telemetry information and said transmitter transmits said telemetry information in a telemetry signal.

7. The system of claim 6, wherein said telemetry information includes at least one of a pH reading, a temperature reading, a pressure, a stress measurement, a strain measurement, and a pulse.

8. The system of claim 1, wherein said transmitter also transmits an identity signal, said identity signal including at least one of an identity of said device, an identity of a patient, an identity of a manufacturer of said device, and an identity of a type of said device.

9. The system of claim 6, wherein said transmitter transmits said position and telemetry signals in a cyclic order.

10. The system of claim 8, wherein said transmitter transmits said position and identity signals in a cyclic order.

11. A method for integrating a sensor into a medical device including:
connecting a transmitter to a medical device to minimize a distance between a tracking point of said device and said transmitter;
transmitting a position signal from said transmitter; and
receiving said position signal at a receiver.

12. The method of claim 11, wherein said connecting step includes embedding said transmitter in said medical device.

13. The method of claim 11, further including measuring said position signal and determining at least one of a position and orientation of said transmitter relative to said receiver.

14. The method of claim 11, wherein said position signal includes at least one of a unique identifier and telemetry signal.

15. The method of claim 11, wherein said transmitter is an electromagnetic coil and said receiver is an array of electromagnetic coils.

16. The method of claim 11, further including:
measuring additional telemetry information at said transmitter; and
transmitting said telemetry information in a telemetry signal from said transmitter.

17. The method of claim 16, wherein said telemetry information includes at least one of a pH reading, a temperature reading, a pressure, a stress measurement, a strain measurement, and a pulse.

18. The method of claim 11, wherein said transmitting step includes transmitting an identity signal, said identity signal including at least one of an identity of said device, an identity of a patient, an identity of a manufacturer of said device, and an identity of a type of said device.

19. The method of claim 16, wherein said transmitting said position signal and transmitting said telemetry signal occur in a cyclic order.

20. The method of claim 18, wherein said transmitting said position signal and transmitting said identity signals occur in a cyclic order.

21. A method for locating a medical device in a patient and providing a device characteristic, said method including:
connecting a transmitter to said medical device;
transmitting a signal from said transmitter; and
receiving said signal at a receiver, said signal including at least one of a position and orientation of said transmitter relative to said receiver and said device characteristic.

22. The method of claim 21, wherein said transmitting step includes transmitting at least one of said position and orientation and said device characteristic in a multiplexed fashion.
